(19) United States
(12) Patent Application Publication
(10) Pub. No.: US 2022/0015941 A1
(43) Pub. Date: Jan. 20, 2022

(54) Wearable Icepack Holding Apparatus

(71) Applicant: Nic Lamb, Manhattan Beach, CA (US)

(72) Inventor: Nic Lamb, Manhattan Beach, CA (US)

(21) Appl. No.: 17/305,729

(22) Filed: Jul. 14, 2021

Related U.S. Application Data
(60) Provisional application No. 63/052,361, filed on Jul. 15, 2020.

(51) Int. Cl. A61F 7/02; A61F 7/10

(52) U.S. Cl. CPC A61F 7/02; A61F 2007/0008; A61F 7/10

(57) ABSTRACT

A wearable icepack holding apparatus is disclosed. The wearable icepack holding apparatus comprises a body and one or more fastening means. The body is configured to be worn on a user's head. The body comprises a first pocket at a first portion of the body. Further, the body comprises a plurality of second pockets at one or more second portions of the body. Each of the first pocket and the plurality of second pockets is configured to hold one or more icepacks. Further, the one or more fastening means are attached at a third portion of the body and are configured to adjust a grip of the body on the user's head.

### FIELD OF THE DISCLOSURE

The invention relates to a wearable apparatus. More particularly, the invention relates to the wearable apparatus for covering a user's head for performing hypothermia therapy, to reduce headache occurs due to fatigue, migraine, and many other similar problems.

### BACKGROUND OF THE DISCLOSURE

The subject matter discussed in this background section should not be assumed to be prior art merely as a result of its mention herein. Similarly, any problems mentioned in this background section or associated with the subject matter of this background section should not be assumed to have been previously recognized in the prior art. The subject matter as disclosed in this background section merely represents different approaches related to apparatuses for hypothermia therapy, wherein such apparatuses themselves may also correspond to implementations of the claimed technology and invention.

In today's world, most of the people are living a stressful life, which may result in depression, hypertension, and other issues related to mental health. Such issues further result in mild and severe headaches. Typically, people use ointments, balms, and painkiller medicines, in order to get rid of severe headaches. The ointments and balms reduce the headache but these are harmful to skin. On the other hand, the painkiller medicines are not good for health as they do have side effects or allergic reactions on people's bodies. Further, an incorrect dosage of painkiller medicines can cause severe side effects on the health of a person, which may require immediate medical attention.

Further, people used to wrap their head tightly with a piece of cloth, to reduce the headache or pain that occurs due to migraine or other issues related to mental health. However, wrapping the head tightly is not recommended by doctors as it reduces the blood circulation in the head/brain. In order to overcome this problem, icepacks are introduced. Icepacks are headcovers with plastic bags that contain fluids at a low temperature. The fluid is selected based on its ability to retain low temperature for a long duration of time. However, a user of the icepack needs to hold the icepack on the aching area, and thus it is inconvenient and difficult to hold the icepacks for a long duration of time. Therefore, in order to overcome the problem of holding the icepacks for a long duration of time, apparatuses were introduced to fix or hold the icepacks at the head, which is aching.

Prior art, for various aspects contained therewithin, relevant to this disclosure includes U.S. design Pat. No. 5,92,836 to Woo-Cho, U.S. Pat. Publication No 2016/0058084 to Stevenson, U.S. Pat. Publication No. 2018/0303182 to Smith, and U.S. design Pat. No. 8,72,292 to Marcus. In each of these prior arts, an apparatus is provided to be used for holding an icepack on the user's head and the like. This is not an ideal solution to the problem of fixing icepacks over the head of the user for providing relaxation to the user.

In particular, reference '836 to Woo-Cho discloses a cap with a band-shaped cooling pack. The cap consists of a space where an icepack is placed. After inserting the icepack in the space provided in the cap, the user needs to wear the cap. The icepack inside the cap provides relief on forehead of the user. However, unlike the subject matter of the disclosed invention, Woo-Cho does not discuss or suggest the aspect of using multiple icepacks. Further, the cap does not consist of pockets to carry more than one icepack. Further, Woo-Cho does not disclose the aspect of using fastening means for adjusting the grip of the cap on the user's head. Furthermore, Woo-Cho is silent on the aspect of design of icepacks.

Reference '084 to Stevenson discloses a headband with removable cooling elements. The headband consists of multiple pockets to hold a number of cooling elements and is made of stretchable material so that the user may wear it easily. However, unlike the subject matter of the disclosed invention, Stevenson does not discuss or suggest the aspect that a cooling element is provided on an upper side of the user's head. Further, Stevenson does not disclose the aspect of using fastening means for adjusting the grip of the headband on the user's head. Furthermore, Stevenson is silent on the aspect of the design of icepacks.

Reference '182 to Smith discloses a temperature control head covering system, with a knit cap. The knit cap houses an icepack, in a temperature controlled liner. The icepack is placed at a temperature control liner, to provide cooling on the upper part of the head of the user. However, unlike the subject matter of the disclosed invention, Smith does not discuss or suggest the aspect of placement of multiple icepacks on sides of the knit cap, apart from the temperature control liner. Moreover, Smith does not disclose the aspect of using fastening means for adjusting the grip of the knit cap on the user's head. Furthermore, Smith is silent on the aspect of the design of icepacks.

Reference '292 to Marcus discloses a cooling gel pack placed in a cap; the cooling gel pack is folded to adjust inside the cap. However, unlike the subject matter of the disclosed invention, Marcus does not discuss or suggest the aspect of using multiple icepacks in multiple portions of the cap. Further, the prior art is silent about the adjustment of the cap according to the size of the user's head, by using fastening means. Furthermore, Marcus is silent on the aspect of the design of icepacks.

Given the deficiencies of the prior arts, the need remains for an effective and improved apparatus to cover the user's head and perform hypothermia therapy for providing relief from headache and protect the users from intake of heavy dosage of medicines.

### SUMMARY OF THE DISCLOSURE

According to embodiments illustrated herein, a novel, simple and inexpensive wearable icepack holding apparatus is disclosed. The wearable icepack holding apparatus comprises a body configured to be worn on a user's head. The body comprises a first pocket at a first portion of the body and a plurality of second pockets at one or more second portions of the body. The first portion corresponds to an upper side of the body and the one or more second portions correspond to at least a left side and a right side of the body. Further, each of the first pocket and the plurality of second pockets is configured to hold one or more icepacks. The wearable icepack holding apparatus further comprises one or more fastening means attached at a third portion of the body. The third portion corresponds to a rear side of the body. The one or more fastening means are selected from a group comprising of an adjusting strip, an adjusting pad, a zip, a VELCRO, or a hook-and-loop fastener. The one or more fastening means are configured to adjust a grip of the body on the user's head. Such adjustment of the grip provides comfort to the user and facilitates an improved hold of the body on the user's head.

In one embodiment, the first pocket and the plurality of second pockets having one or more locking means such as a zip or a hook-and-loop fastener, to prevent the one or more icepacks from falling out of the first pocket and the plurality of second pockets. Further, each icepack of the one or more icepacks is made of a material selected from a group comprising of at least a plastic or a polymer. Further, each icepack of the one or more icepacks comprises at least a fluid configured to retain low temperature that helps in decreasing pain of the user's head and reduces intake of medicines. Further, each icepack of the one or more icepacks includes a plurality of slits for conforming the one or more icepacks to the user's head.

In one alternate embodiment herein, a wearable icepack holding apparatus is disclosed. The wearable icepack holding apparatus comprises a body configured to be worn on a user's head. The body comprises a first pocket at a first portion of the body. The first portion corresponds to an upper side of the body. The first pocket is configured to hold a first icepack. The first icepack having an elongated rectangular shape and a plurality of slits to conform the first icepack to the user's head. The first icepack helps in decreasing pain of the user's head and reduces intake of medicines. Further, the body comprises a plurality of second pockets at one or more second portions of the body. The one or more second portions correspond to at least a left side and a right side of the body. Further, each of the plurality of second pockets is configured to hold one or more second icepacks. The wearable icepack holding apparatus further comprises one or more fastening means attached at a third portion of the body. The third portion corresponds to a rear side of the body. The one or more fastening means are configured to adjust a grip of the body on the user's head. Such adjustment of the grip provides comfort to the user and facilitates an improved hold of the body on the user's head.

In one embodiment, the first pocket and the plurality of second pockets having one or more locking means such as a zip or a hook-and-loop fastener, to prevent the one or more icepacks from falling out of the first pocket and the plurality of second pockets. Further, the first icepack and each icepack of the one or more second icepacks is made of a material selected from a group comprising of at least a plastic or a polymer. Further, the one or more second icepacks include a semicircular portion. It can be noted that the semicircular portion covers sides of the user's head. Further, the one or more second icepacks include a rectangular portion. It can be noted that the rectangular portion is attached to the semicircular portion. Further, the rectangular portion covers a backside of the user's head. Further, the one or more second icepacks include a plurality of slits to conform the one or more second icepacks to the user's head. In one embodiment, the first icepack and each icepack of the one or more second icepacks comprise at least a fluid configured to retain low temperature that helps in decreasing pain of the user's head and reduces intake of medicines. Further, the fluid is made at least of compounds selected from a group of propylene glycol, hydroxyethyl cellulose, or vinyl-coated silica gel. Locking and plastic

In one embodiment, the body is made of a material selected from a group of materials including a soft micro fiber polyester, a fabric, a stretchable material, or a combination of fabrics. Further, the material is a washable material.

Other features and aspects of this disclosure will be apparent from the following description and the accompanying drawings.

### BRIEF DESCRIPTION OF THE DRAWINGS

While the specification concludes with claims particularly pointing out and distinctly claiming particular embodiments of the present disclosure, various embodiments of the present disclosure can be more readily understood and appreciated from the following descriptions of various embodiments of the present disclosure when read in conjunction with the accompanying drawings, in which:

FIG. 1A illustrates a front top perspective view of a wearable icepack holding apparatus, according to an embodiment of the present disclosure;

FIG. 1B illustrates a top view of the wearable icepack holding apparatus, according to an embodiment of the present disclosure;

FIG. 1C illustrates a rear view of the wearable icepack holding apparatus, according to an embodiment of the present disclosure;

FIG. 1D illustrates a left side view of the wearable icepack holding apparatus, according to an embodiment of the present disclosure;

FIG. 1E illustrates a right side view of the wearable icepack holding apparatus, according to an embodiment of the present disclosure;

FIG. 2 illustrates a perspective view of a first icepack, according to an embodiment of the present disclosure; and

FIG. 3 illustrates a perspective view of a second icepack, according to another embodiment of the present disclosure.

### DETAILED DESCRIPTION

Reference will now be made in detail to specific embodiments or features, examples of which are illustrated in the accompanying drawings. Wherever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems, and methods are now described.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

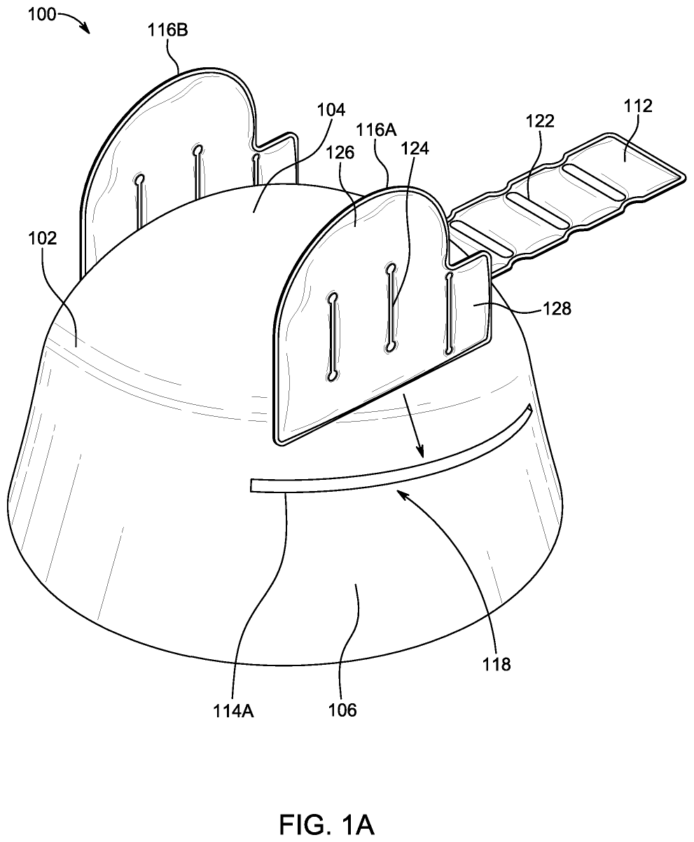

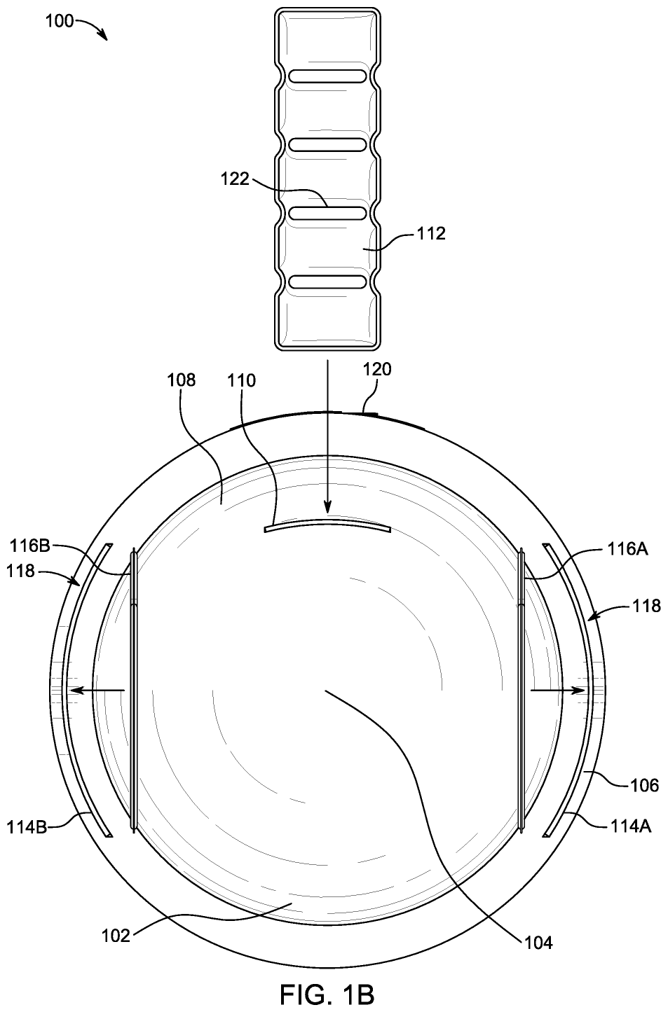

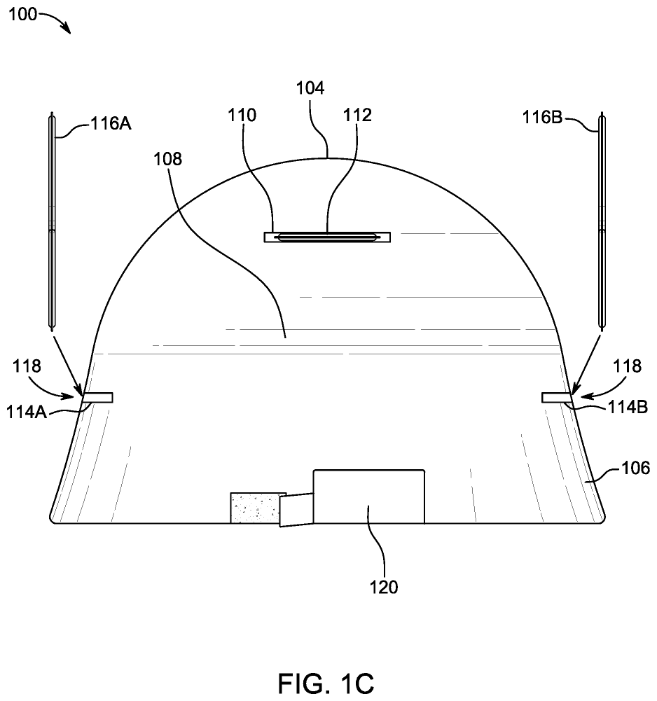

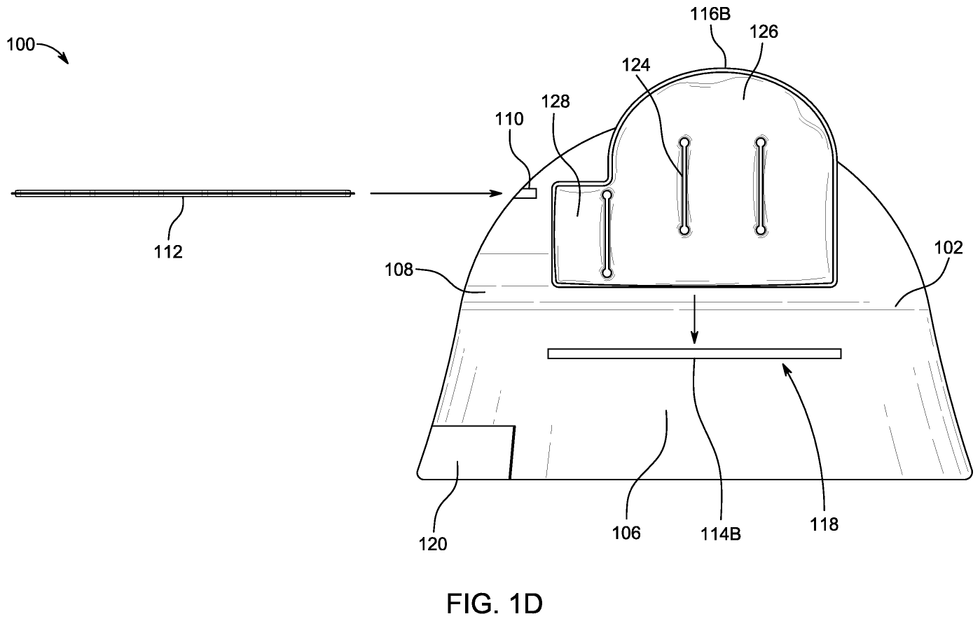

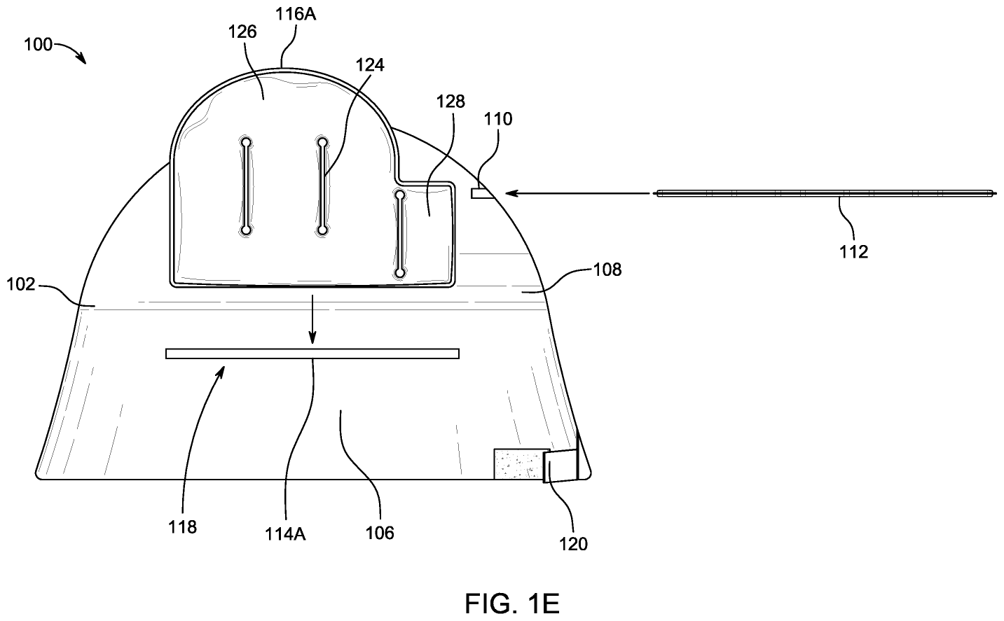

FIG. 1A illustrates a front top perspective view of a wearable icepack holding apparatus 100, according to an embodiment of the present disclosure. FIG. 1A is described in conjunction with FIGS. 1B-1E.

The wearable icepack holding apparatus 100 may comprise a body 102. The body 102 having a first portion 104, one or more second portions 106, and a third portion 108. In one embodiment, the first portion 104 may correspond to an upper side i.e. top side of the body 102 (as shown in FIG. 1B). The one or more second portions 106 may correspond to at least a left side and a right side of the body 102 (as shown in FIG. 1A). The third portion 108 may correspond to a rear side of the body 102 (as shown in FIG. 1B). It can be noted that the above-mentioned sides of the first portion 104, the one or more second portions 106, and the third portion 108, without departing from the scope of the disclosure.

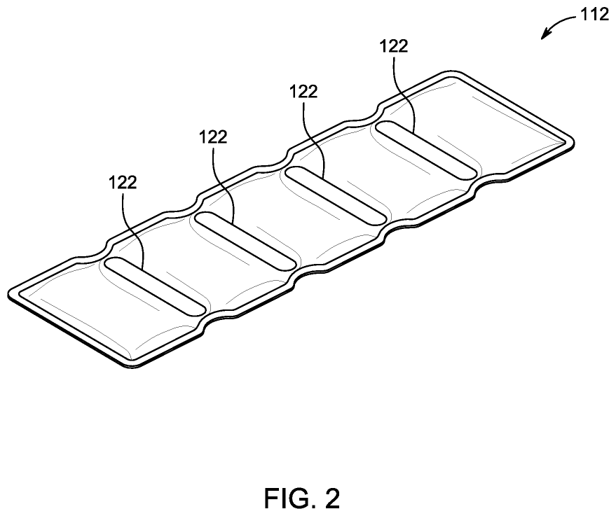

Further, the body 102 having a first pocket 110 at the first portion 104. Further, the first pocket 110 having a pre-defined size and dimension. In one example embodiment, the first pocket 110 is rectangular in shape. It can be noted that the first pocket 110 may be enclosed between an inner surface and an outer surface of the body 102. Further, the first pocket 102 may be configured to hold a first icepack 112. In one embodiment, the first icepack 112 may be able to reach till a forehead area of the body 102, to provide a cooling effect to a user's forehead. Further, the first icepack 112 may be filled with fluid such as a gel. It can be noted that the fluid may be configured to retain low temperature, and thus helps in decreasing pain of the user's head and reduces intake of medicines. In an exemplary embodiment, the gel substance is comprised of polyvinyl chloride (PVC), water, carboxymethyl cellulose (CMC), glycerinum, and antiseptic. In one aspect, the gel substance ratio is 0.3 mm PVC, 70% water, 4% CMC, 25% glycerinum, and 1% antiseptic. The detailed description of the first icepack 112 may be described later in conjunction with FIG. 2.

Further, the body 102 may have a plurality of second pockets 114A, 114B at the one or more second portions 106. The plurality of second pockets 114A, 114B may have a pre-defined size and dimension. In one example embodiment, the plurality of second pockets 114A, 114B are semicircular in shape. It can be noted that the plurality of second pockets 114A, 114B may be enclosed between an inner surface and an outer surface of the body 102. Further, the plurality of second pockets 114A, 114B may be configured to hold one or more second icepacks 116A, 116B respectively. In one embodiment, a second pocket 114A may be at the left side of the body 102. It can be noted that the second pocket 114A may be configured to hold a second icepack 116A. In another embodiment, the second pocket 114B may be at the right side of the body 102. It can be noted that the second pocket 114B may be configured to hold a second icepack 116B.

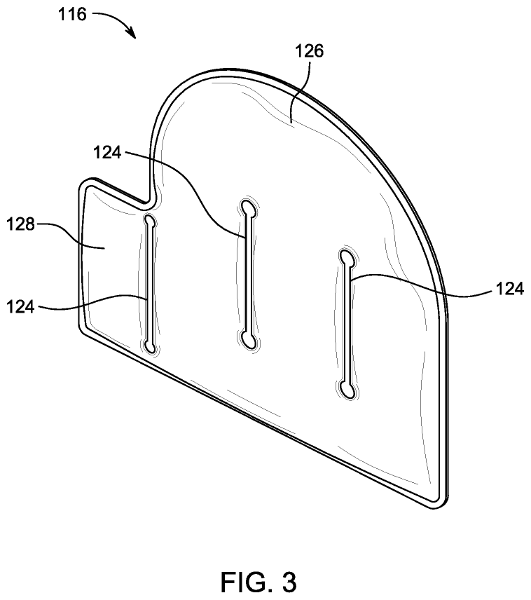

Further, the one or more second icepacks 116A, 116B may be filled with fluid such as a gel. It can be noted that the fluid may be configured to retain low temperature, and thus helps in decreasing pain of the user's head and reduces intake of medicines. The detailed description of the one or more second icepacks 116A, 116B may be described later in conjunction with FIG. 3. It will be apparent to one skilled in the art that the first pocket 110 and the plurality of second pockets 114A, 114B have been provided only for illustration purpose. In one embodiment, the body 102 may be provided with a fewer or greater number of pockets, without departing from the scope of the disclosure.

As shown in FIGS. 1A-1E, the first pocket 110 and the plurality of second pockets 114A, 114B may include one or more locking means 118. In one embodiment, the one or more locking means 118 may be a zip or a hook-and-loop fastener. It can be noted that the one or more locking means 118 may prevent one or more icepacks i.e. the first icepack 112 and the one or more second icepacks 116A, 116B from falling out of the first pocket 110 and the plurality of second pockets 114A, 114B respectively.

Further, the wearable icepack holding apparatus 100 may comprise one or more fastening means 120 attached at the third portion 108 of the body 102. As discussed above, the third portion 108 may correspond to the rear side of the body 102. The one or more fastening means 120 may be, but not limited to, an adjusting strip, an adjusting pad, a zip, a VELCRO, or a hook-and-loop fastener. It can be noted that the adjustment of the one or more fastening means 120 may be based at least on the user's head size. In one exemplary embodiment, the adjusting strip may be positioned on an outer surface of the body 102 to couple to an adjacent adjusting pad. In another exemplary embodiment, the one or more fastening means 120 may be coupled to the body 102 by stitching the one or more fastening means 120 to the body 102 or by using an adhesive. The one or more fastening means 120 may be configured to adjust a grip of the body 102 on the user's head. Such adjustment of the grip provides comfort to the user and facilitates an improved hold of the body 102 on the user's head.

Further, the one or more fastening means 120 may facilitate a precise fitting of the wearable icepack holding apparatus 100 atop the user's head. Such precise fit of the wearable icepack holding apparatus 100 may provide effective cooling to the user during and/or after an athletic activity in which the user engages, and thus reduces overheating and decreases the likelihood of heat stress and/or heatstroke, which can result from the exercise and/or ambient temperature/humidity.

In one embodiment, the wearable icepack holding apparatus 100 may be a cap, a hat, or a hoodie. In one example embodiment, the wearable icepack holding apparatus 100 is a cap (as shown in FIG. 1A). The body 102 of the wearable icepack holding apparatus 100 may be configured to be worn on the user's head. In one embodiment, the body 102 may be made from synthetic and/or manmade materials, natural materials, and/or blended combinations thereof. The materials may be, but not limited to, acrylic, nylon, spandex, soft micro fiber polyester, or cotton. In another embodiment, the body 102 may be made of a combination of nylon and spandex. It should be noted that material of the body 102 may be a washable material. Such usage of the above-mentioned materials provides excellent stretchability for wearing the wearable icepack holding apparatus 100 and conforming the wearable icepack holding apparatus 100 on the user's head.

Further, at least an interior side (or layer) of the body 102 may also incorporate a material, or a combination of materials (e.g., cotton or a cotton blend), which can absorb and/or draw away perspiration from the user's head. The wearable icepack holding apparatus 100 may facilitate in curing migraines, sleep insomnia, head injuries, recovery/performance, and well-being. The wearable icepack holding apparatus 100 may be configured to provide a combination of cooling and comfort to the user. It will be apparent to one skilled in the art that the above-mentioned materials have been provided only for illustration purposes, without departing from the scope of the disclosure.

FIGS. 2 and 3 illustrate a perspective view of a first icepack 112 and a second icepack 116, according to an embodiment of the present disclosure. Hereinafter, the second icepack 116 may correspond to either of the one or more second icepacks 116A and 116B. FIGS. 2 and 3 are described in conjunction with FIGS. 1A-1E.

As shown in FIG. 2, the first icepack 112 may include a plurality of slits 122. The plurality of slits 122 may facilitate the first icepack 112 to retain an original shape. Further, the plurality of slits 122 may prevent the first icepack 112 from disfiguring. In one embodiment, the plurality of slits 122 may allow the fluid inside the first icepack 112 to be in uniform shape and thus assist in efficient use of the wearable icepack holding apparatus 100. Further, the plurality of slits 122 may assist in conforming the first icepack 112 to different shapes of the user's head. In addition, the plurality of slits 122 may allow the first icepack 112 to remain flexible and stay around the topside of the user's head.

Further, as discussed above, the first icepack 112 may be able to reach forehead area of the body 102, to provide the cooling effect to the user's forehead. Further, the first icepack 112 may be filled with a fluid such as a gel. It can be noted that the fluid may be configured to retain low temperature, and thus helps in decreasing pain of the user's head and reduces intake of medicines. Further, the fluid inside the first icepack 112 may have the tendency to repel heat and a pre-defined temperature inside the first icepack 112. In one embodiment, the first icepack 112 may be removable from the first pocket 110, without departing from the scope of the disclosure.

As shown in FIG. 3, the second icepack 116 may correspond to either of the one or more second icepacks 116A and 116B, discussed above. Further, the second icepack 116 may include a plurality of slits 124. The plurality of slits 124 may facilitate the second icepack 116 to retain an original shape. Further, the plurality of slits 124 may prevent the second icepack 116 from disfiguring. In one embodiment, the plurality of slits 124 may allow the fluid inside the second icepack 116 to be in uniform shape, and thus assist in efficient use of the wearable icepack holding apparatus 100. Further, the plurality of slits 124 may assist in conforming the second icepack 116 to different shapes of the user's head. In addition, the plurality of slits 124 may allow the second icepack 116 to remain flexible and stay around the left side and right side of the user's head. It can be noted that shape of the second icepack 116 may be formed due to the presence of the plurality of slits 124.

Further, the second icepack 116 having a semicircular portion 126 and a rectangular portion 128 attached to the semicircular portion 126. The semicircular portion 126 of the second icepack 116 may assist in covering the sides of the user's head. On the other hand, the rectangular portion 128 may facilitate covering a backside of the user's head or neck area of the user. Further, the second icepack 116 may facilitate cooling sides i.e. left side and right side of the user's head. Further, as discussed above, the second icepack 116 may be filled with a fluid such as a gel. It can be noted that the fluid may be configured to retain low temperature, and thus helps in decreasing pain of the user's head and reduces intake of medicines. Further, the fluid inside the second icepack 116 may have the tendency to repel heat and a pre-defined temperature inside the second icepack 116. In one embodiment, the second icepacks 116A, 116B may be removable from the plurality of second pockets 114A, 114B, without departing from the scope of the disclosure.

In one embodiment, the wearable icepack holding apparatus 100 may be stored, for cooling for use, in a box filled with layers of dry ice to sandwich the wearable icepack holding apparatus 100. In one embodiment, the wearable icepack holding apparatus 100 may be maintained within a temperature range of −18 to −30 degree Celsius. In an example embodiment, the following temperatures (as shown in Table 1) are recorded after storing the wearable icepack holding apparatus 100 in the box, which is layered with evenly distributed 1 inch of dry ice at base of the box, then placing the wearable icepack holding apparatus 100, followed by evenly distributing 1 inch of dry ice layered on top of the wearable icepack holding apparatus 100. It can be noted that the wearable icepack holding apparatus 100 is removed from the box and temperature is recorded by inserting a thermometer for 6 minutes within the wearable icepack holding apparatus 100, at recurring intervals of 6 minutes. In one embodiment, the wearable icepack holding apparatus 100 may become rigid. Further, the temperature reading may be better after 60 minutes on dry ice by leaving the thermometer in the wearable icepack holding apparatus 100 for a longer duration than 6 minutes.

TABLE 1

| Time | Temperature |
|---|---|
| (minutes) | (Celsius) |
| 0 | Ambient |
| 30 | 0 |
| 60 | −15 |
| 90 | −30 |

In one embodiment, the fluid may be a combination of multiple compounds in a pre-defined proportion. In one exemplary embodiment, a composition of the fluid is 0.3 mm Polyvinyl chloride (PVC) +70% water+4% carboxymethyl cellulose (CMC) +25% glycerinum+1% antiseptic substance. In another embodiment, the fluid may be high grade specialized polyurethane. In another embodiment, the fluid inside the one or more icepacks i.e. the first icepack 112 and the second icepack 116 may be made of compounds selected from a group of propylene glycol, hydroxyethyl cellulose, or vinyl-coated silica gel. Further, the propylene glycol is a synthetic organic compound that is a viscous colorless liquid and does not have any odor. The chemical formula for propylene glycol is CH₃CH(OH)CH₂OH. Further, the propylene glycol is soluble in a wide range of solvents like chloroform, acetone, or water. It can be noted that the use of the propylene glycol is prominent due to the heat exchange capabilities.

Further, the hydroxyethyl cellulose is an agent that is derived from cellulose and is a thickening and gelling compound. The chemical formula of hydroxyethyl cellulose is C₃₆H₇₀O₁₉. It can be noted that hydroxyethyl cellulose has the capability to provide structural strength to the one or more icepacks. Further, hydroxyethyl cellulose and alongside the propylene glycol, may assist in maintaining the one or more icepacks in a state where the one or more icepacks can be reused multiple times without failure.

Further, the one or more icepacks may be made of a plastic material or polymer. In one embodiment, the one or more icepacks may be stored in cool places like refrigerator before inserting into the body 102. It can be noted that the low temperature of the refrigerator decreases the temperature of the one or more icepacks. Further, after the use, the one or more icepacks may be placed in the refrigerator, to cool the fluid inside the one or more icepacks and can be reused. In one embodiment, the first icepack 112 and the second icepacks 116A, 116B may be removable from the first pocket 110 and the plurality of second pockets 114A, 114B respectively. It should be noted that the above-mentioned material of the one or more icepacks has been provided only for illustration purposes, without departing from the scope of the disclosure.

In one embodiment, the wearable icepack holding apparatus 100 may be reused multiple times. Further, the reuse of the wearable icepack holding apparatus 100 facilitates economically to the user, as the user can use the same wearable icepack holding apparatus 100 for multiple times. Further the wearable icepack holding apparatus 100 may facilitate the user to perform any other task while wearing the wearable icepack holding apparatus 100 and there is no need to stay at one place upon wearing the wearable icepack holding apparatus 100. Thus, the wearable icepack holding apparatus 100 may help the user in reducing the pain in a natural way by treating therapeutic hypothermia. Further, the wearable icepack holding apparatus 100 may cool down the temperature of the user's head and provide relief from mild and severe headaches.

In an exemplary embodiment, there is a study table below disclosing the results and feedbacks obtained by the users of the wearable icepack holding apparatus 100.

| | | | MEDICINE |
|---|---|---|---|
| USER | PROBLEM | RESULT | INTAKE |
| Customer 1 | Mild Headache | Relief in very less | Without any medicine |
| | | time | |
| Customer 2 | Severe Headache | Relief in an hour | Without any medicine |
| Customer 3 | Migraine | Relief in few hours | Taken in very less |
| | | | quantity |
| Customer 4 | Sports injury on head | Relief in swelling in | Taken medicine only |
| | | very less time | for the wound |
| Customer 5 | headache due to | Relief in very less | Without any medicine |
| | Hangover | time | |
| Customer 6 | Headache due to | Relief in very less | Without any medicine |
| | stress | time | |
| Customer 7 | Headache due to high | Relief in an hour | Without any medicine |
| | fever | | |
| Customer 8 | Headache due to | Relief in very less | Without any medicine |
| | fatigue | time | |
| Customer 9 | Sleeping insomnia | Relief in very less | Without any medicine |
| | | time | |

As shown in the above mentioned table, it should be noted that the wearable icepack holding apparatus 100 reduces the amount of medicine intake by the users. Further, the wearable apparatus 100 helps the user in reducing the headache without taking any painkiller medicines. The medicines used as painkillers may be very harmful for the user's health as they may have many side effects, most of the doctors' advice not to take painkiller. Further, the use of painkillers may damage kidney and create many other problems like increase in cardiovascular risk, poor concentration, memory problem, constipation, slow breathing rate, slow reaction and movement, apathy, and alike.

It will be apparent to one skilled in the art that the above-mentioned components of the wearable icepack holding apparatus 100 have been provided only for illustration purposes. Further, embodiments of the present invention relate to an article of a headgear that, upon installation of the wearable icepack holding apparatus 100 turns into a protective helmet, hard hat or other headgear, that may keep the user's head both cool and cushioned from the interior harness of the headgear, without departing from the scope of the disclosure.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A wearable icepack holding apparatus comprising:
a body configured to be worn on a user's head, wherein the body having:
a first pocket at a first portion of the body; and
a plurality of second pockets at one or more second portions of the body,
wherein each of the first pocket and the plurality of second pockets is configured to hold one or more icepacks; and
one or more fastening means attached at a third portion of the body, the one or more fastening means are configured to adjust a grip of the body on the user's head.

2. The wearable icepack holding apparatus of claim 1, wherein the first pocket and the plurality of second pockets, having one or more locking means such as a zip or a hook-and-loop fastener, to prevent the one or more icepacks from falling out of the first pocket and the plurality of second pockets.

3. The wearable icepack holding apparatus of claim 1, wherein the first portion corresponds to an upper side of the body.

4. The wearable icepack holding apparatus of claim 1, wherein the one or more second portions correspond to at least a left side and a right side of the body.

5. The wearable icepack holding apparatus of claim 1, wherein the third portion corresponds to a rear side of the body.

6. The wearable icepack holding apparatus of claim 1, wherein the one or more fastening means are selected from a group comprising of an adjusting strip, an adjusting pad, a zip, a VELCRO, or a hook-and-loop fastener.

7. The wearable icepack holding apparatus of claim 1, wherein the adjustment of the one or more fastening means is based at least on the user's head size.

8. The wearable icepack holding apparatus of claim 1, wherein each icepack of the one or more icepacks is made of a material selected from a group comprising of at least a plastic or a polymer.

9. The wearable icepack holding apparatus of claim 1, wherein each icepack of the one or more icepacks comprises at least a fluid configured to retain low temperature that helps in decreasing pain of the user's head and reduces intake of medicines.

10. The wearable icepack holding apparatus of claim 1, wherein each icepack of the one or more icepacks include a plurality of slits for conforming the one or more icepacks to the user's head.

11. The wearable icepack holding apparatus of claim 1, wherein each icepack of the one or more icepacks in the first pocket is rectangular in shape, and each icepack of the one or more icepacks in the plurality of second pockets is semicircular in shape.

12. The wearable icepack holding apparatus of claim 1, wherein an icepack in the first pocket is smaller than each of the icepacks in the plurality of second pockets.

13. The wearable icepack holding apparatus of claim 1, wherein the body is made of a material selected from a group of materials including a soft micro fiber polyester, fabric, stretchable material, or a combination of fabrics, and the material is a washable material.

14. A wearable icepack holding apparatus comprising:
a body configured to be worn on a user's head, wherein the body having:
a first pocket at a first portion of the body, wherein the first pocket is configured to hold a first icepack, the first icepack having an elongated rectangular shape and a plurality of slits to conform the first icepack to the user's head; and
a plurality of second pockets at one or more second portions of the body, wherein each of the plurality of second pockets are configured to hold one or more second icepacks; and
one or more fastening means attached at a third portion of the body, the one or more fastening means are configured to adjust a grip of the body on the user's head.

15. The wearable icepack holding apparatus of claim 14, wherein each of the one or more second icepacks include:
a semicircular portion covers sides of the user's head; and
a rectangular portion attached to the semicircular portion, wherein the rectangular portion covers a backside of the user's head.

16. The wearable icepack holding apparatus of claim 14, wherein the one or more second icepacks include a plurality of slits to conform the one or more second icepacks to the user's head.

17. The wearable icepack holding apparatus of claim 14, wherein the first icepack and each icepack of the one or more second icepacks comprise at least a fluid configured to retain low temperature that helps in decreasing pain of the user's head and reduces intake of medicines.

18. The wearable icepack holding apparatus of claim 17, wherein the fluid is made at least of compounds selected from a group of propylene glycol, hydroxyethyl cellulose, or vinyl-coated silica gel.

19. The wearable icepack holding apparatus of claim 14, wherein the first icepack and each of the one or more second icepacks are made of a plastic material or polymer.

20. The wearable icepack holding apparatus of claim 14, wherein the first pocket and the plurality of second pockets, include one or more locking means such as a zip or a hook-and-loop fastener, to prevent icepacks from falling out of the first pocket and the plurality of second pockets.
